(19) United States
(12) Patent Application Publication
(10) Pub. No.: US 2023/0338279 A1
(43) Pub. Date: Oct. 26, 2023

(54) NASAL SPRAY FORMULATION WITH MOISTURIZING BENEFITS

(71) Applicant: GLAXOSMITHKLINE CONSUMER HEALTHCARE HOLDINGS (US) LLC, Wilmington, DE (US)

(72) Inventors: Venkata Vijay Kumar AKUNURI, Mississauga, Ontario (CA); Pallav Arvind BULSARA, Warren, NJ (US); Maneshkumar P. PATEL, Warren, NJ (US)

(21) Appl. No.: 17/918,790

(22) PCT Filed: Apr. 13, 2021
(86) PCT No.: PCT/US2021/026974

§ 371 Date: Oct. 13, 2022

Related U.S. Application Data
(60) Provisional application No. 63/009,660, filed on Apr. 14, 2020.

(51) Int. Cl. A61K 9/00; A61K 47/10; A61K 47/26; A61K 31/56

(52) U.S. Cl. CPC A61K 9/0043; A61K 47/10; A61K 47/26; A61K 31/56

(57) ABSTRACT

Aspects of the present invention are directed to a nasal spray formulation comprising: at least one active pharmaceutical ingredient, glycerin, polyethylene glycol, and dextrose. Formulations of the present invention provide moisturizing benefits and retain at least about 3% water when exposed to about 80% relative humidity at a temperature of about 23° C. for about 750 minutes in a Dynamic Vapor Sorption (DVS) test.

### TECHNICAL FIELD

The present invention relates to nasal spray formulations with at least one active pharmaceutical ingredient and at least one moisturizing agent.

### BACKGROUND

Nasal dryness and the associated discomfort in the nasal cavity is a common complaint of patients who suffer from allergic rhinitis. In a study, at least 41% of allergy sufferers reported experiencing dryness in their nose as an associated symptom of allergic rhinitis or a side effect of the most common allergy symptom treatments, including nasal sprays. Currently marketed nasal sprays, including FLONASE® brand nasal sprays sold by GlaxoSmithKline Consumer Healthcare, treat the symptoms of allergic rhinitis but do not significantly address nasal dryness. Dryness is perceived to be at least as much of a concern as other symptoms experienced by allergy sufferers.

Dryness can cause sore skin inside the nasal passages which can lead to bleeding from the nose or blisters. Furthermore, there may be a burning sensation, particularly when blowing or rubbing the nose. Dryness is not limited to the skin inside the nasal passage and may even visibly affect the outside of the nose and surrounding areas between the nose and lips. There is an unmet need in the market for a moisturizing nasal spray which alleviates nasal dryness and provides a moisturizing sensation in addition to treating the symptoms of allergic rhinitis.

One of the major hinderances to adding a moisturizing agent to a nasal spray formulation is that the addition of a moisturizing agent may adversely affect the availability of the active pharmaceutical ingredient at the deposited site, making the formulation less effective at treating the symptoms of allergic rhinitis. Additionally, the moisturizing agent may affect the osmolarity, rheology, and/or pH of the formulation which could lead to irritation or affect the stability of the formulation.

Based on the above, it is desirable to develop a nasal spray formulation that is effective at treating the symptoms of allergic rhinitis while also alleviating nasal dryness.

### SUMMARY

An embodiment of the present invention is directed to a nasal spray formulation comprising: at least one active pharmaceutical ingredient; glycerin; polyethylene glycol; and dextrose; wherein the formulation retains at least about 3% water when exposed to about 80% relative humidity at a temperature of about 23° C. for about 750 minutes.

Another embodiment of the present invention is directed to a nasal spray formulation comprising: at least one active pharmaceutical ingredient, glycerin; polyethylene glycol; and dextrose.

In an embodiment, formulations of the present invention have a pH between about 5 and about 7. In an embodiment, formulations have an osmolarity between about 100 mOsmoles and about 800 mOsmoles.

In an embodiment, the active pharmaceutical ingredient in formulations is present in an amount between about 0.005% w/w and about 0.2% w/w. In an embodiment, the active pharmaceutical ingredient is fluticasone propionate, fluticasone furoate, azelastine, oxymetazoline, xylometazoline, beclomethasone, mometasone, budesonide, salts and esters thereof, or a combination thereof. In a preferred embodiment, the active pharmaceutical ingredient is fluticasone propionate.

In another embodiment, formulations of the present invention are isotonic and include an isotonicity adjusting agent being sodium chloride, dextrose, potassium chloride, or a combination thereof. In an embodiment, the isotonicity adjusting agent is sodium chloride.

In an embodiment, glycerin is present in formulations of the present invention in an amount between about 0.5% w/w and about 8% w/w. In an embodiment, dextrose is present in an amount between about 0.3% w/w and about 7% w/w. In an embodiment, polyethylene glycol is present in an amount between about 0.5% w/w and about 20% w/w. In an embodiment, the polyethylene glycol has an average molecular weight between about 200 and about 600. In a preferred embodiment, the polyethylene glycol has an average molecular weight of about 400.

In an embodiment, formulations of the present invention have a droplet size distribution of about 10% of droplets are less than about 18 μm, about 50% of droplets are between about 33 μm and about 51 μm, and about 90% of droplets are below about 120 μm. In an embodiment, about 100% of the active pharmaceutical ingredient particles are less than about 10 μm.

An embodiment of the present invention is directed to a method of treating the symptoms of allergic rhinitis whereby the nasal cavity feels moisturized, comprising administering the formulation according to the invention. An embodiment of the present invention is directed to a method of treating the symptoms of allergic rhinitis whereby a gently soothing is provided in the nasal cavity, comprising administrating the formulation according to the invention. An embodiment of the present invention is directed to a method of treating the symptoms of allergic rhinitis whereby the nasal cavity feels comfortable, comprising administering the formulation according to the invention.

### DETAILED DESCRIPTION

Aspects of the present invention are directed to nasal spray formulations including at least one active pharmaceutical ingredient suitable for nasal administration and a moisturizing agent or a combination of moisturizing agents. Applicants have recognized that, surprisingly, these nasal spray formulations soothe dryness and provide a moisturizing sensation without adversely affecting the stability, efficacy or safety of the formulations.

Formulations of the present invention include at least one active pharmaceutical ingredient or a combination of active pharmaceutical ingredients. In an embodiment, the active pharmaceutical ingredient is for the treatment of inflammatory and allergic conditions, including seasonal and perennial allergic rhinitis. Suitable active pharmaceutical ingredients include, corticosteroids, antihistamines, alpha adrenergic agonists, among others. Examples of an active pharmaceutical ingredient that may be included in formulations of the present invention include, but are not limited to, fluticasone, azelastine, oxymetazoline, xylometazoline, beclomethasone, mometasone, budesonide, chlorpheniramine maleate, loratadine, azatadine, salts and esters thereof, or a combination thereof. The amount of the active pharmaceutical ingredient may be between about 0.001% w/w and about 0.5% w/w based on the total weight of the formulation.

In a preferred embodiment, the active pharmaceutical ingredient is fluticasone propionate. The amount of fluticasone propionate may be between about 0.005% w/w and about 0.2% w/w based on the total weight of the formulation. In an embodiment, the amount of fluticasone propionate may be between about 0.01% w/w and about 0.09% w/w based on the total weight of the formulation. In a preferred embodiment, the amount of fluticasone propionate is about 0.05% w/w based on the total weight of the formulation.

In another embodiment, the active pharmaceutical ingredient is fluticasone furoate. The amount of fluticasone furoate may be between about 0.005% w/w and about 0.2% w/w based on the total weight of the formulation. In an embodiment, the amount of fluticasone furoate may be between about 0.01% w/w and about 0.09% w/w based on the total weight of the formulation. In a preferred embodiment, the amount of fluticasone furoate is about 0.05% w/w based on the total weight of the formulation.

For example, a composition according to the invention to be administered by dispensing from a multiple use metered nasal spray container may comprise fluticasone propionate in an amount sufficient to provide 0.05 mg/spray; or fluticasone furoate in an amount sufficient to provide 0.0275 mg/spray; or azelastine or its pharmaceutically acceptable salt (e.g., azelastine hydrochloride) in an amount sufficient to provide the equivalent of 0.125 mg of base per spray, wherein the total number of spray counts per container is, e.g., 50, 60, 120 or 144 spray counts.

In an embodiment, the formulation may be isotonic and include an isotonicity adjusting agent in place of the active pharmaceutical ingredient or in combination with the active pharmaceutical ingredient. Examples of isotonicity adjusting agents include, but are not limited to, sodium chloride, dextrose, potassium chloride, or a combination thereof.

In a preferred embodiment, the isotonicity adjusting agent is sodium chloride. The amount of sodium chloride may be between about 0.1% w/w and about 1.5% w/w based on the total weight of the formulation. In an embodiment, the amount of sodium chloride may be between about 0.5% w/w and about 1.3% w/w based on the total weight of the formulation. In a preferred embodiment, the amount of sodium chloride is about 0.9% w/w based on the total weight of the formulation.

In another embodiment, the isotonicity adjusting agent is dextrose. The amount of dextrose may be between about 1% w/w and about 10% w/w based on the total weight of the formulation. In an embodiment, the amount of dextrose may be between about 3% w/w and about 7% w/w based on the total weight of the formulation. In a preferred embodiment, the amount of dextrose is about 5% w/w based on the total weight of the formulation.

Formulations of the present invention include a moisturizing agent or a combination of moisturizing agents. The moisturizing agent or combination of moisturizing agents may provide a moisturizing sensation and/or alleviate dryness in the nasal cavity and/or surrounding areas. Typically, the addition of any moisturizing agent may negatively impact the stability of the formulation, availability of the active pharmaceutical ingredient in the formulation, and/or safety of the formulation, among other characteristics. Applicants have recognized that, surprisingly, the addition of a particular combination of moisturizing agents in formulations of the present invention provide moisturizing benefits while also maintaining characteristics including, but not limited to, pH, droplet size distribution (DSD), particle size distribution (PSD), dissolution, and osmolarity within the desired ranges to maintain the stability, efficacy, and safety of the formulations.

A significant advantage of the formulations of the present invention compared to FLONASE® is that although the formulations of the present invention include moisturizing agents, these formulations are still bioequivalent to currently approved and marketed FLONASE® which does not include any moisturizing agents. This is significant because the formulations of the present invention can treat symptoms of allergic rhinitis substantially as effectively and safely as FLONASE® while also providing additional moisturizing benefits.

As used herein, bioequivalence is the absence of a significant difference in the rate and extent to which the active pharmaceutical ingredient becomes available at the site of drug action when administered at the same dose under similar conditions in an appropriately designed study. A drug product containing the same active ingredient in the same amount as another drug product, is considered to be bioequivalent to the approved drug product if the rate and extent of absorption does not show a significant difference from the approved drug product, or the extent of absorption does not show a significant difference and any difference in rate is intentional or not medically significant.

For formulations of the present invention, bioequivalence may depend on the DSD and the PSD of the active pharmaceutical ingredient, among other factors. The rate and extent to which the active pharmaceutical ingredient becomes available at the site of action for formulations of the present invention is substantially similar to FLONASE®, making the formulations bioequivalent to FLONASE®.

Suitable moisturizing agents that may be included in formulations of the present invention include, but are not limited to, glycerin, propylene glycol, hyaluronic acid sodium, DL lactic acid, polyvinyl pyrrolidine, polyethylene glycol, or a combination thereof. In an embodiment, the moisturizing agent may be glycerin, polyethylene glycol having an average molecular weight between about 200 and about 600, propylene glycol, or a combination thereof.

In a preferred embodiment, a combination of moisturizing agents is included in formulations of the present invention, wherein the combination is of glycerin and polyethylene glycol with an average molecular weight of 400 (PEG400). Although not intended to be limited to a single theory, PEG400 is believed to promote moisturization in the nasal cavity by increasing water retention and providing a soothing effect. Further, glycerin is believed to add to the moisturizing benefit by drawing water from the air into the skin's outer layer and by forming a protective layer that helps prevent moisture loss.

The amount of PEG400 may be between about 0.5% w/w and about 20% w/w based on the total weight of the formulation. In an embodiment, PEG400 may be included in an amount between about 1% w/w and about 5% w/w based on the total weight of the formulation. In a preferred embodiment, the amount of PEG400 is about 2% w/w based on the total weight of the formulation.

The amount of glycerin may be between about 0.5% w/w and about 8% w/w based on the total weight of the formulation. In an embodiment, the amount of glycerin may be between about 1% w/w and about 4% w/w based on the total weight of the formulation. In a preferred embodiment, the amount of glycerin is about 2.5% w/w based on the total weight of the formulation.

In a preferred embodiment, the combination of moisturizing agents is a combination of about 2% w/w of PEG400 based on the total weight of the formulation and about 2.5% w/w of glycerin based on the total weight of the formulation.

As the amount of moisturizing agents in the formulations of the present invention increases, characteristics of the formulations may deviate further from FLONASE®. It has been recognized that including a combination of moisturizing agents as opposed to a single moisturizing agent provides a moisturizing effect without unduly increasing the total amount of moisturizing agents in the formulations. Further, the use of a combination of moisturizing agents provides better water retention and maintains the desired characteristics of the formulations better as compared to a single moisturizing agent.

The nasal cavity is a sensitive area and nasal spray formulations preferably should have an osmolarity close to bodily fluids. An osmolarity adjuster may be included in formulations of the present invention to maintain the osmolarity of the formulations within the desired range. Examples of suitable osmolarity adjusters include, but are not limited to, sodium chloride, dextrose and calcium chloride. In a preferred embodiment, the osmolarity adjuster is dextrose, most preferably used as dextrose anhydrous.

The addition of moisturizing agents in formulations of the present invention may impact the osmolarity by increasing it to the point of possible irritation. Therefore, the amount of dextrose used in the formulations of the present invention should be suitable to maintain an appropriate osmolarity.

The amount of dextrose may be between about 0.03% w/w and about 7% w/w based on the total formulation. In an embodiment, the amount of dextrose may be between about 1% w/w and about 2.5% w/w based on the total weight of the formulation. In a preferred embodiment, the amount of dextrose is about 2% w/w based on the total weight of the formulation.

The osmolarity of formulations of the present invention may be between about 100 mOsmoles and about 800 mOsmoles. In an embodiment, the osmolarity may be between about 300 mOsmoles and about 600 mOsmoles. In a preferred embodiment, the osmolarity is about 454 mOsmoles.

Formulations of the present invention may have a water content between about 80% w/w and about 99% w/w based on the total weight of the formulation. In an embodiment, the water content may be between about 85% w/w and about 97% w/w based on the total weight of the formulation. In a preferred embodiment, the water content is between about 90% w/w and about 95% w/w based on the total weight of the formulation.

A Dynamic Vapor Sorption (DVS) instrument can be used to assess the formulations' ability to retain water and provide moisturizing benefits. The instrument includes a micro balance where samples of microliter scale (i.e. 8-12 microliters, e.g., 10 μl) are loaded into a pan. The pan is enclosed in a chamber where the temperature and humidity are controlled. As the sample is exposed to constant humidity, the weight loss over time is captured by the microbalance.

The formulations tested initially contain about 90% w/w to about 95% w/w water based on the total weight of the formulation. The rate of water loss as captured by weight loss via the microbalance is recorded by the instrument. The relative humidity (RH) was maintained at about 80% for an initial 750 minutes after which the RH was dropped to about 20% and maintained for a subsequent 750 minutes. The temperature was maintained at about 23° C. for the entirety of the test.

In an embodiment, the DVS results indicate at least about 3% water retained during the initial 750 minutes. In another embodiment, the DVS results indicate greater than about 3% water retained during the initial 750 minutes. In another embodiment, the DVS results indicate between about 3% and about 10% water retained during the initial 750 minutes. In another embodiment, the DVS results indicate between about 3.5% and about 7% water retained during the initial 750 minutes. In a preferred embodiment, the water retention according to the DVS results is about 5.82% water retained during the initial 750 minutes. Comparatively, FLONASE® has a water retention of about 3% water retained as measured by DVS during the initial 750 minutes. In an embodiment, formulations of the present invention retained at least about 50% more water than FLONASE® during the initial 750 minutes. In another embodiment, formulations of the present invention retained at least about 75% more water than FLONASE® during the initial 750 minutes.

The RH was dropped to about 20% for a subsequent 750 minutes of the DVS test. In an embodiment, the DVS results indicate between about 0.3% and about 1.5% water retained during the subsequent 750 minutes. In another embodiment, the DVS results indicate between about 0.5% and about 0.9% water retained during the subsequent 750 minutes. In a preferred embodiment, the DVS results indicate about 1% water retained during the subsequent 750 minutes. Comparatively, FLONASE® has a water retention between about 0.15% and about 0.3% water retained during the subsequent 750 minutes. In an embodiment, formulations of the present invention retained at least about 50% more water than FLONASE® during the subsequent 750 minutes. In another embodiment, formulations of the present invention retained at least about 75% more water than FLONASE® during the subsequent 750 minutes. In another embodiment, formulations of the present invention retained at least about 100% more water than FLONASE® during the subsequent 750 minutes.

Preferably, the formulations of the present invention maintain a pH between about and about 7 for the duration of their self-life. It was observed that adding only glycerin in an amount to be effective as a moisturizing agent increased the pH beyond the acceptable range. Further, adding only PEG400 in an amount to be effective as a moisturizing agent decreased the pH beyond the acceptable range. Applicants have recognized that including a combination of glycerin and PEG400 provides a pH within the acceptable range while also providing moisturizing benefits. Certain embodiments of the present invention have a pH between about 5 and about 7 throughout their shelf-life.

Formulations of the present invention are preferably sprayed as a fine mist and droplets are deposited topically. The droplets are then absorbed and become available locally to treat the symptoms of allergic rhinitis. It is believed that the availability of the active pharmaceutical ingredient at the deposited site impacts the efficacy and safety of the formulations. The availability of the active pharmaceutical ingredient is a function of DSD and active pharmaceutical ingredient PSD inside the droplets of the formulations.

The viscosity of the formulations may affect the DSD. Glycerin may increase the viscosity of the formulations resulting in a potentially larger droplet size distribution. In contrast, PEG400 may decrease the viscosity of the formulations resulting in a potentially smaller droplet size distribution. Applicants have recognized that a particular combination of glycerin and PEG400 balances the viscosity and rheology profile to allow for the appropriate DSD. Preferably, the DSD and the PSD of the formulations of the present invention may be substantially similar to FLONASE® so that the safety and efficacy is consistent.

The DSD of formulations of the present invention may be as follows: about 10% of the droplets are less than about 25 μm, about 50% of the droplets are between about 25 μm and about 60 μm, and about 90% of the droplets are below about 150 μm. In another embodiment, the DSD may be as follows: about 10% of the droplets are less than about 20 μm, about 50% of the droplets are between about 30 μm and about 55 μm, and about 90% of the droplets are below about 130 μm. In a preferred embodiment, the DSD is as follows: about 10% of the droplets are less than about 18 μm, about 50% of the droplets are between about 33 μm and about 51 μm, and about 90% of the droplets are below about 120 μm.

In an embodiment, at time zero (Tzero), the DSD is as follows: about 10% of the droplets are less than about 18 μm, about 50% of the droplets are between about 33 μm and about 51 μm, and about 90% of the droplets are below about 120 μm.

Tzero is the time at which the formulation is placed in a controlled environment for stability testing. In an embodiment, Tzero is within about 60 days after the manufacturing process is complete. In another embodiment, Tzero is within about 7 days after the manufacturing process is complete.

In an embodiment, after storage for 1 month at 40° C./75% RH, the DSD is as follows: about 10% of the droplets are less than about 18 μm, about 50% of the droplets are between about 33 μm and about 51 μm, and about 90% of the droplets are below about 120 μm.

In an embodiment, after storage for 3 months at 40° C./75% RH, the DSD is as follows: about 10% of the droplets are less than about 18 μm, about 50% of the droplets are between about 33 μm and about 51 μm, and about 90% of the droplets are below about 120 μm.

In an embodiment, after storage for 6 months at 40° C./75% RH, the DSD is as follows: about 10% of the droplets are less than about 18 μm, about 50% of the droplets are between about 33 μm and about 51 μm, and about 90% of the droplets are below about 120 μm.

In an embodiment, the DSD of formulations of the present invention is within at least about 20% of the DSD of FLONASE®. In an embodiment, the DSD of formulations of the present invention is within at least about 20% of the DSD of FLONASE® at Tzero, after storage for 1 month at 40° C./75% RH, after storage for 3 months at 40° C./75% RH, and after storage for 6 months at 40° C./75% RH. The DSD of formulations of the present invention supports that the formulations are bioequivalent to FLONASE®.

The formulations of the present invention were further tested using Morphologically Directed Raman Spectroscopy (MDRS). MDRS is a technique to measure the PSD of active pharmaceutical ingredient particles. This technique distinguishes the active pharmaceutical ingredient particles from particles of excipients and enables the measurement of particles specific to the active pharmaceutical ingredient.

The PSD of the active pharmaceutical ingredient for formulations of the present invention may be as follows: about 50% to about 75% of the active pharmaceutical ingredient particles are less than about 2 μm, about 85% to about 99% of the active pharmaceutical ingredient particles are less than about 3 μm, about 95% to about 100% of the active pharmaceutical ingredient particles are less than about 5 μm and about 97% to about 100% of the active pharmaceutical ingredient particles as less than about 10 μm.

In an embodiment, at Tzero, the PSD of the active pharmaceutical ingredient is as follows: about 66% of the active pharmaceutical ingredient particles are less than about 2 μm, about 95% of the active pharmaceutical ingredient particles are less than about 3 μm, about 100% of the active pharmaceutical ingredient particles are less than about 5 μm, and about 100% of the active pharmaceutical ingredient particles are less than about 10 μm.

The PSD for FLONASE® at Tzero is as follows: about 60% of the active pharmaceutical ingredient particles are less than about 2 μm, about 95% of the active pharmaceutical ingredient particles are less than about 3 μm, about 100% of the active pharmaceutical ingredient particles are less than about 5 μm, and about 100% of the active pharmaceutical ingredient particles are less than about 10 μm.

After storage for 1 month at 40° C./75% RH, in an embodiment, the PSD of the active pharmaceutical ingredient is as follows: about 61% of the active pharmaceutical ingredient particles are less than about 2 μm, about 95% of the active pharmaceutical ingredient particles are less than about 3 μm, about 100% of the active pharmaceutical ingredient particles are less than about 5 μm, and about 100% of the active pharmaceutical ingredient particles are less than about 10 μm.

The PSD for FLONASE® after storage for 1 month at 40° C./75% RH, is as follows: about 49% of the active pharmaceutical ingredient particles are less than about 2 μm, about 86% of the active pharmaceutical ingredient particles are less than about 3 μm, about 100% of the active pharmaceutical ingredient particles are less than about 5 μm, and about 100% of the active pharmaceutical ingredient particles are less than about 10 μm.

After storage for 3 months at 40° C./75% RH, in an embodiment, the PSD is as follows: about 65% of the active pharmaceutical ingredient particles are less than about 2 μm, about 94% of the active pharmaceutical ingredient particles are less than about 3 μm, about 100% of the active pharmaceutical ingredient particles are less than about 5 μm, and about 100 percent of the active pharmaceutical ingredient particles are less than about 10 μm.

The PSD for FLONASE®, after storage for 3 months at 40° C./75% RH, is as follows: about 50% of the active pharmaceutical ingredient particles are less than about 2 μm, about 85% of the active pharmaceutical ingredient particles are less than about 3 μm, about 99% of the active pharmaceutical ingredient particles are less than about 5 μm, and about 100% of the active pharmaceutical ingredient particles are less than about 10 μm.

In an embodiment, the PSD of active pharmaceutical ingredient particles is within at least about 20% of the PSD of active pharmaceutical ingredient particles for FLONASE®. In an embodiment, the PSD of active pharmaceutical ingredient particles is within at least 20% of the PSD of active pharmaceutical ingredient particles for FLONASE® at Tzero, after storage for 1 month at 40° C./75% RH, and after storage for 3 months at 40° C./75% RH. The PSD of active pharmaceutical ingredient particles of formulations of the present invention supports that the formulations are bioequivalent to FLONASE®.

The formulations of the present invention were further studied for their dissolution rate of active pharmaceutical ingredient particles using an in-vitro dissolution technique. PSD may have an impact on dissolution rate and absorption, i.e. larger particles may dissolve slower than smaller particles, affecting availability of the active pharmaceutical ingredient at the site of deposition.

The test was performed for samples stored at 40° C./75% RH at Tzero, 1 month, and 3 months. In an embodiment, the dissolution profile showed that the rate of dissolution of the active pharmaceutical ingredient in formulations of the present invention is substantially similar to the dissolution of the active pharmaceutical ingredient in FLONASE®.

Formulations of the present invention may further include a suspending agent. Examples of suspending agents include, but are not limited to, carboxymethylcellulose, veegum, tragacanth, bentonite, methylcellulose and polyethylene glycols, or a combination thereof.

In a preferred embodiment, the suspending agent is microcrystalline cellulose and carboxy methylcellulose sodium, preferably used as the branded product AVICEL® RC-591 or AVUCEL® CL-611. AVICEL® RC-591 typically contains about 87% to about 91% microcrystalline cellulose and about 9% to about 13% sodium carboxymethylcellulose.

Microcrystalline cellulose and carboxy methylcellulose sodium may be present in an amount between about 0.5% w/w and about 5% w/w based on the total weight of the formulation. In an embodiment, microcrystalline cellulose and carboxy methylcellulose sodium may be present in an amount between about 1% w/w and about 3% w/w based on the total weight of the formulation. In a preferred embodiment, the amount of microcrystalline cellulose and carboxy methylcellulose sodium is about 1.5% w/w based on the total weight of the formulation.

Formulations of the present invention may further include preservatives to protect the formulation from contamination and microbial growth. Examples of preservatives include, but are not limited to, quaternary ammonium compounds (benzalkonium chloride, benzethonium chloride, cetrimide and cetylpyridinium chloride), mercurial agents (e.g. phenylmercuric nitrate, phenylmercuric acetate and thimerosal), alcoholic agents (e.g. chlorobutanol, phenylethyl alcohol and benzyl alcohol), antibacterial esters (e.g. esters of parahydroxybenzoic acid), chelating agents such as disodium edetate (EDTA) and other anti-microbial agents such as cholorhexidine, chlorocresol, sorbic acid and its salts and polymyxin, or a combination thereof.

In an embodiment, the preservatives are benzalkonium chloride, phenylethyl alcohol, or a combination thereof. In a preferred embodiment, the preservatives are a combination of benzalkonium chloride and phenylethyl alcohol.

The amount of benzalkonium chloride may be between about 0.005% w/w and about 0.2% w/w based on the total weight of the formulation. In an embodiment, the amount of benzalkonium chloride may be between about 0.01% w/w and about 0.09% w/w based on the total weight of the formulation. In a preferred embodiment, the amount of benzalkonium chloride is about 0.02% w/w based on the total weight of the formulation.

The amount of phenylethyl alcohol may be between about 0.05% w/w and about 0.5% w/w based on the total weight of the formulation. In an embodiment, the amount of phenylethyl alcohol may be between about 0.15% w/w and about 0.35% w/w based on the total weight of the formulation. In a preferred embodiment, the amount of phenylethyl alcohol is about 0.25% w/w based on the total weight of the formulation.

The present invention further includes a wetting agent. Examples of suitable wetting agents include, but are not limited to, fatty alcohols, esters, ethers, or a combination thereof. In a preferred embodiment, the wetting agent is polyoxyethylene (2) sorbitan monooleate, preferably as the branded product POLYSORBATE® 80.

The amount of polyoxyethylene (2) sorbitan monooleate may be between about 0.0005% w/w and about 0.09% w/w based on the total weight of the formulation. In an embodiment, the amount of polyoxyethylene (2) sorbitan monooleate may be between about 0.001% w/w and about 0.01% w/w based on the total weight of the formulation. In a preferred embodiment, the amount of polyoxyethylene (2) sorbitan monooleate is about 0.005% w/w based on the total weight of the formulation.

Formulations of the present invention may be used with a delivery device including a pump to facilitate topical administration to the nasal cavity by means of a metered atomizing spray pump. The pump may be designed to deliver about 100 mg (100 μl) of suspension per actuation. The delivery device preferably delivers an equal dose with equal DSD and spray pattern per activation. In a preferred embodiment, to ensure uniformity of the dosage, a homogenous dispersion of the active ingredient is achieved by the use of micronized fluticasone propionate, pre-wetted with polyoxyethylene (2) sorbitan monooleate, in combination with the thixotropic suspending agent, microcrystalline cellulose and carboxymethylcellulose sodium (AVICEL® RC591).

Formulations of the present invention may be administered topically to the nasal cavity of a person in need of such treatment by spraying the formulations of the present invention using the delivery device. In an embodiment, the present invention may be administered to the nasal cavity to treat the symptoms of allergic rhinitis, including seasonal and perennial rhinitis, inflammatory conditions, asthma, COPD, dermatitis, among others. Examples of symptoms include, but are not limited to, nasal congestion, sneezing, watery eyes, itchy eyes, itchy nose, runny nose, or a combination thereof.

In an embodiment, formulations of the present invention may make the nasal cavity feel moisturized, soothed, comfortable, or a combination thereof. Further, formulations of the present invention may provide relief to the nasal cavity from irritation, dryness, discomfort, or a combination thereof.

### EXAMPLES

### Examples 1-6—Examples of Nasal Spray Formulations

Embodiments of the present invention may be prepared as indicated below in Examples 1-5. In a preferred embodiment, the nasal spray formulation is prepared according to Example 1. Example 6 illustrates the formulation of FLONASE®.

TABLE 1
Examples of Nasal Spray Formulations

| | | Amount (% w/w) | | | | | |
|---|---|---|---|---|---|---|---|
| Ingredient | Function | Ex. 1 | Ex. 2 | Ex. 3 | Ex. 4 | Ex. 5 | Ex. 6 |
| Fluticasone | Active | 0.05 | 0.05 | 0.05 | 0.05 | 0.05 | 0.05 |
| Propionate | | | | | | | |
| Micronised | | | | | | | |
| Dextrose | Osmolarity | 2 | 2 | 2.5 | 2.5 | 0 | 5 |
| Anhydrous | agent | | | | | | |
| Glycerin | Moisturizing | 2.5 | 1.5 | 1.75 | 1.75 | 4 | 0 |
| USP | agent | | | | | | |
| Polyethylene | Moisturizing | 2 | 3 | 4 | 0 | 0 | 0 |
| Glycol 400 NF | agent | | | | | | |
| Propylene | Moisturizing | 0 | 0.75 | 0 | 0.75 | 0 | 0 |
| Glycol | agent | | | | | | |
| Phenylethyl | Preservative | 0.25 | 0.25 | 0.25 | 0.25 | 0.25 | 0.25 |
| Alcohol USP | | | | | | | |
| Avicel | Suspending | 1.5 | 1.5 | 1.5 | 1.5 | 1.5 | 1.5 |
| RC591 (GI) | agent | | | | | | |
| Benzalkonium | Preservative | 0.02 | 0.02 | 0.02 | 0.02 | 0.02 | 0.02 |
| Chloride | | | | | | | |
| EP/USNF/JP | | | | | | | |
| Polysorbate | Wetting | 0.005 | 0.005 | 0.005 | 0.005 | 0.005 | 0.005 |
| 80 (GI) | agent | | | | | | |
| Purified Water | Vehicle | 100 | 100 | 100 | 100 | 100 | 100 |
| Calculated | NA | 5.71 | 6.03 | 6.6 | 4.8 | 5.2 | 3 |
| Water | | | | | | | |
| Content (% | | | | | | | |
| water retained) | | | | | | | |
| Observed | NA | 5.82 | 6.43 | 7.02 | 4.73 | NA | 3 |
| Water | | | | | | | |
| Content (% | | | | | | | |
| water retained) | | | | | | | |
| Calculated | NA | 454 | 469 | 451 | 449 | 299 | 299 |
| Osmolarity | | | | | | | |
| (mOsmoles) | | | | | | | |

The observed water content is determined using DVS. The calculated water content is calculated using Design-Expert® Software to create a predictive statistical model. The observed water content data was plotted using the Design-Expert® Software to create a design space with the known concentrations of glycerin, PEG400, and dextrose as the inputs and the measured water content values as the outputs.

### Example 7—Stability and Spray Characteristics of the Nasal Spray Formulations

The stability and spray characteristics of certain embodiments of the present invention were tested at Tzero, at 3 months storage at 40° C./75% RH and at 6 months storage at 40° C./75% RH.

The results of the stability tests are shown in tables 2-5 below. The study generally concluded that formulations of the present invention complied with the stability specifications for all time points and storage conditions.

The results of the spray characteristics tests are shown in tables 6-9 below. The spray characteristics tests generally concluded that formulations of the present invention complied with the spray characteristics specifications for all time points and storage conditions.

TABLE 2
Stability Data - Tzero

| | pH |
|---|---|
| Example 1 | 6.3 |
| Example 2 | 6.3 |
| Example 3 | 6.3 |
| Example 4 | 6.3 |
| Example 5 | 6.3 |
| Example 6 | 6.2 |

TABLE 3
Stability Data - Storage for 1 Month at 40° C./75% RH

| | pH |
|---|---|
| Example 1 | 6.0 |
| Example 2 | 5.7 |
| Example 3 | 4.8 |
| Example 4 | 6.2 |

TABLE 4
Stability Data - Storage for 3 Months at 40° C./75% RH

| | pH |
|---|---|
| Example 1 | 5.8 |
| Example 2 | 5.5 |
| Example 5 | 7.9/9.0 |
| Example 6 | 5.6 |

TABLE 5
Stability Data - Storage for 6 months at 40° C./75% RH

| | pH |
|---|---|
| Example 1 | 5.5 |
| Example 2 | 5.3 |
| Example 6 | 5.1 |

TABLE 6
Spray Characteristics—Tzero

| | Plume | | | | | | | | | | | |
|---|---|---|---|---|---|---|---|---|---|---|---|---|
| | Geometry | | Spray Pattern | | | | Droplet Size Distribution | | | | | |
| Formu- | Spray | Plume | D | D | Ratio | | D10 | D50 | D75 | D90 | | % droplet |
| lation | Angle | Width | Min | Max | (<1.7) | Area | (<18 μ) | (33-51) | (NA) | (<120) | Span | <10 μ (4%) |
| Ex. 1 | 51.7 | 58.6 | 13.0 | 18.2 | 1.387 | 196.8 | 15.56 | 42.10 | 69.52 | 101.42 | 2.043 | 2.776 |
| Ex. 2 | 47.2 | 52.5 | 12.1 | 16.6 | 1.389 | 159.0 | 16.06 | 44.62 | 73.42 | 106.65 | 2.040 | 2.326 |
| Ex. 3 | 48.3 | 54.0 | 13.2 | 16.8 | 1.275 | 173.0 | 16.20 | 44.77 | 73.61 | 106.25 | 2.018 | 2.253 |
| Ex. 4 | 49.0 | 54.9 | 11.3 | 14.4 | 1.278 | 125.7 | 15.71 | 43.12 | 71.31 | 103.37 | 2.034 | 2.398 |
| Ex. 5 | 54.6 | 62. | 11.3 | 14.6 | 1.290 | 135.2 | 14.97 | 37.73 | 62.7 | 91.76 | 2.034 | 2.617 |
| Ex. 6 | 51.8 | 58.6 | 11.3 | 14.6 | 1.303 | 127.6 | 15.81 | 42.25 | 69.39 | 101.35 | 2.028 | 2.450 |

TABLE 7
Spray Characteristics—Storage for 1 Month at 40° C./75% RH

| | Plume | | | | | | | | | | | |
|---|---|---|---|---|---|---|---|---|---|---|---|---|
| | Geometry | | Spray Pattern | | | | Droplet Size Distribution | | | | | |
| Formu- | Spray | Plume | D | D | Ratio | | D10 | D50 | D75 | D90 | | % droplet |
| lation | Angle | Width | Min | Max | (<1.7) | Area | (<18 μ) | (33-51) | (NA) | (<120) | Span | <10 μ (4%) |
| Ex. 1 | 56.5 | 64.6 | 14.1 | 20.3 | 1.4 | 222.0 | 14.29 | 37.31 | 61.40 | 90.72 | 2.050 | 3.748 |
| Ex. 2 | 60.7 | 70.8 | 14.1 | 19.6 | 1.4 | 214.2 | 13.49 | 34.73 | 57.08 | 85.04 | 2.060 | 4.533 |
| Ex. 3 | 58.9 | 67.9 | 15.2 | 19.3 | 1.3 | 234.2 | 12.8 | 32.50 | 53.14 | 79.81 | 2.060 | 5.359 |
| Ex. 4 | 57.7 | 66.4 | 13.8 | 17.7 | 1.3 | 195.7 | 12.98 | 31.76 | 51.28 | 76.24 | 1.989 | 4.850 |
| Ex. 5 | 55.7 | 63.5 | 14.3 | 16.9 | 1.180 | 192.9 | 14.36 | 36.98 | 60.98 | 90.31 | 2.057 | 3.508 |
| Ex. 6 | 57.4 | 66.0 | 14.9 | 19 | 1.278 | 227.6 | 13.97 | 35.97 | 57.01 | 84.18 | 2.007 | 4.068 |

TABLE 8
Spray Characteristics—Storage for 3 Months at 40° C./75% RH

| | Plume | | | | | | | | | | | |
|---|---|---|---|---|---|---|---|---|---|---|---|---|
| | Geometry | | Spray Pattern | | | | Droplet Size Distribution | | | | | |
| Formu- | Spray | Plume | D | D | Ratio | | D10 | D50 | D75 | D90 | | % droplet |
| lation | Angle | Width | Min | Max | (<1.7) | Area | (<18 μ) | (33-51) | (NA) | (<120) | Span | <10 μ (4%) |
| Ex. 1 | 65.2 | 76.8 | 13.5 | 18.7 | 1.396 | 196.3 | 13.49 | 33.22 | 53.84 | 80.36 | 2.013 | 4.330 |
| Ex. 2 | 68.8 | 82.2 | 14.6 | 18.4 | 1.245 | 222.5 | 13.79 | 35.03 | 57.28 | 85.20 | 2.041 | 4.169 |
| Ex. 5 | 52.5 | 59.3 | 11.9 | 15.2 | 1.290 | 136.9 | 14.37 | 35.99 | 58.92 | 87.4 | 2.032 | 3.396 |
| Ex. 6 | 54.8 | 62.4 | 13.9 | 17.9 | 1.292 | 198.8 | 14.12 | 35.59 | 57.31 | 83.37 | 1.946 | 3.716 |

TABLE 9
Spray Characteristics—Storage for 6 Months at 40° C./75% RH

| | Plume | | | | | | | | | | | |
|---|---|---|---|---|---|---|---|---|---|---|---|---|
| | Geometry | | Spray Pattern | | | | Droplet Size Distribution | | | | | |
| Formu- | Spray | Plume | D | D | Ratio | | D10 | D50 | D75 | D90 | | % droplet |
| lation | Angle | Width | Min | Max | (<1.7) | Area | (<18 μ) | (33-51) | (NA) | (<120) | Span | <10 μ (4%) |
| Ex. 1 | 66.9 | 79.4 | 16.6 | 20.5 | 1.229 | 274 | 12.46 | 32.05 | 53.16 | 81.79 | 2.174 | 5.882 |
| Ex. 2 | 67.8 | 80.8 | 17.3 | 22.0 | 1.264 | 315 | 12.10 | 30.48 | 49.65 | 70.93 | 2.073 | 6.238 |
| Ex. 6 | 62.6 | 73.6 | 16.7 | 23.2 | 1.380 | 290.1 | 15.10 | 37.42 | 59.37 | 85.42 | 1.882 | 3.146 |

### Example 8—Sensory Consumer Study

The preferred embodiment of the present invention, as detailed in Example 1, was the subject of a sensory consumer study. The procedures and results of the sensory consumer study are presented below.

301 current allergy nasal spray users and those open to using allergy nasal sprays in the future were included in the study. The respondents tested the nasal spray formulation in their home, once per day, for five consecutive days. The respondents completed a computer-based, quantitative online survey each day after using the nasal spray to answer questions about their perception of the sensory attributes of the formulation. The respondents answered questions immediately after use at the same time (+/−30 minutes) each day.

The attributes or questions regarding the nasal spray formulation that were of interest in the study included: 1) the nasal spray makes the inside of my nose feel moisturized; 2) the nasal spray makes me feel a gently soothing sensation inside of my nose; and 3) the nasal spray makes the inside of my nose feel comfortable.

The dose level was a single spray in each nostril per day. The test samples were stored at ambient temperatures and were provided in a plain, unbranded, commercially current packaging. All of the respondents were allergy suffers and dry nose sufferers. The respondents were 54% female and 46% male. The age breakdown of the respondents was as follows: 29% were between 18-34 years old, 24% were between 35-44 years old, 25% were between 45-54 years old, and 23% were between 55-65 years old. 36% of the respondents were nasal spray users and 64% were pill users. The respondents were all in good general health.

The statistical analysis of the data is based on the binomial distribution, which describes the probability of obtaining a number of successes (NS) in a certain number of trials (NT), from which we can calculate p (NS/NT).

The null hypothesis of the test was H₀: p≤80%, indicating that the percentage of respondents feeling the sensation will not exceed the value given by the null hypothesis (in this case 80%). If the estimated value for p is significantly higher than the value given by the null hypothesis, then the null hypothesis is rejected, and it can be concluded that the observed sensation is not due to chance and that p>80%.

The binomial distribution assumes that each separate trial can be described independently by a Bernoulli distribution, with the probability of success being p. In the context of this study, this translates into the assumption that the respondents did not influence each other during the test, and that for each individual respondent, the probability that he/she will feel a sensation is p. The goal of the study is to estimate the value of p and to determine if the estimated value is significant or can be attributed to chance.

### Results:

Section 1—“the Nasal Spray Makes the Inside of My Nose Feel Moisturized”-Immediate Sensation

The Null Hypothesis (H₀) can be rejected and the Alternate Hypothesis (H_{A}) can be accepted. More than 80% of respondents agree with the statement “this nasal spray makes the inside of my nose feel moisturized,” immediately after use on all days and 5 mins after use on Day 1 (see Table 10).

TABLE 10
Results - Inside of Nose Feels Moisturized Immediately After Use

| | “Feels Moisturized” | p-value | Reject H₀ |
|---|---|---|---|
| Day 1 (immediate) | 93% | 0.0000 | YES |
| Day 1 (after 5-mins) | 88% | 0.0002 | YES |
| Day 2 (immediate) | 95% | 0.0000 | YES |
| Day 3 (immediate) | 96% | 0.0000 | YES |
| Day 4 (immediate) | 96% | 0.0000 | YES |
| Day 5 (immediate) | 98% | 0.0000 | YES |

Section 2—“the Nasal Spray Formulation Makes Me Feel a Gently Soothing Sensation Inside of My Nose”—Immediate Sensation

The Null Hypothesis (H₀) can be rejected and the Alternate Hypothesis (H_{A}) can be accepted. More than 80% of respondents agree with the statement “this nasal spray makes me feel a gently soothing sensation inside of my nose,” immediately after use on all days with the exception of 5 minutes after use on Day 1 (see Table 11).

TABLE 11
Results - Formulation Gave the Inside of Nose a
Gently Soothing Sensation Immediately After Use

| | “Gently Soothing Sensation” | P-value | Reject H_{0?} |
|---|---|---|---|
| Day 1 (immediate) | 87% | 0.0009 | YES |
| Day 1 (after 5-mins) | 82% | 0.1673 | NO |
| Day 2 (immediate) | 90% | 0.0000 | YES |
| Day 3 (immediate) | 91% | 0.0000 | YES |
| Day 4 (immediate) | 91% | 0.0000 | YES |
| Day 5 (immediate) | 92% | 0.0000 | YES |

Section 3—“the Nasal Spray Makes the Inside of My Nose Feel Comfortable”—Immediate Sensation

The Null Hypothesis (H₀) can be rejected and the Alternate Hypothesis (H_{A}) can be accepted. More than 80% of respondents agree with the statement “this nasal spray makes the inside of my nose feel comfortable,” immediately after use on all days and 5 mins after use on Day 1 (see Table 12).

TABLE 12
Results - Formulation Made the Inside of Nose
Feel Comfortable Immediately After Use

| | “Feel Comfortable” | P-value | Reject H_{0?} |
|---|---|---|---|
| Day 1 (immediate) | 90% | 0.0000 | YES |
| Day 1 (after 5-mins) | 89% | 0.0000 | YES |
| Day 2 (immediate) | 89% | 0.0000 | YES |
| Day 3 (immediate) | 92% | 0.0000 | YES |
| Day 4 (immediate) | 90% | 0.0000 | YES |
| Day 5 (immediate) | 91% | 0.0000 | YES |

### Conclusion:

Based on the above study, it is valid to conclude that the respondents generally believed that the nasal spray formulation of the present invention makes the inside of their nose feel moisturized immediately after use, the nasal spray formulation makes them feel a gently soothing sensation inside of their nose immediately after use, and the nasal spray makes the inside of their nose feel comfortable immediately after use.

### Example 9: Dissolution Test and Similarly Analysis

The testing was performed following a validated analytical method. A validated analytical method was also followed when evaluating the similarly factor between batches.

The mean profile for each batch tested is shown in Table 13, resulting from averaging the 12 replicates performed for each batch.

TABLE 13
Mean Profile for Each Tested Batch

| Time | XT5V | XT5W | B46J | SC3S | 6P2E | AY5S |
|---|---|---|---|---|---|---|
| (min) | (%) | (%) | (%) | (%) | (%) | (%) |
| 2.5 | 64.74 | 70.11 | 41.54 | 69.08 | 53.55 | 41.40 |
| 5 | 72.40 | 72.72 | 61.54 | 75.78 | 65.49 | 58.31 |
| 10 | 84.36 | 84.09 | 75.13 | 85.99 | 79.58 | 78.94 |
| 15 | 89.27 | 90.14 | 83.23 | 90.94 | 86.23 | 86.17 |
| 20 | 93.35 | 93.23 | 87.45 | 91.81 | 89.29 | 90.12 |
| 25 | 94.24 | 94.67 | 91.12 | 94.92 | 91.86 | 93.18 |
| 30 | 94.78 | 95.70 | 91.76 | 95.58 | 92.03 | 94.87 |
| 60 | 98.28 | 97.54 | 96.37 | 97.86 | 97.07 | 98.54 |
| 120 | 89.98 | 98.65 | 98.52 | 98.81 | 98.35 | 99.91 |
| 180 | 99.41 | 100.02 | 99.08 | 99.14 | 98.55 | 99.48 |
| 240 | 100 | 100 | 100 | 100 | 100 | 100 |

Only one measurement was considered after 85% dissolution of both products; therefore, 4 timepoints were included in the analysis (2.5 min, 5 min, 10 min and 15 min) since the cumulative drug release at 10 minutes was found to be lower than 85% for 5 out of the 6 mean dissolution profiles. Dissolution profiles were compared using the following equation that defines a similarity factor (f2):

f2=50 log 10{[1+1/nΣ(Rt−Tt)2]−0.5×100}

t=1

In the above equation, Rₜ and Tₜ are the percent dissolved at each time point. An f2 value between 50 and 100 suggests the two dissolution profiles are similar. The f2 values resulted from the comparison between each reference product (RP), which is FLONASE®, and test product (TP), which is the preferred embodiment of the present invention detailed in Example 1. This is presented in table 14 below.

TABLE 14
Similarity Factors

| RP batch | TP batch | f2 |
|---|---|---|
| XT5W | SC3S | 83.27 |
| XT5V | 6P2E | 57.04 |
| B46J | AY5S | 75.68 |

All of the similarly factors shown in table 14 suggest that the dissolution profiles of the RP and TP tested are comparable and equivalent.

### Example 10: MDRS Data and Test Results

The data and results below were acquired by Morphologi M4-ID system, using the Morphologically Directed Raman Spectrometry (MDRS) technology. Further, below are the results of the statistical analysis required to assess whether the preferred embodiment of the present invention, as illustrated in Example 1, is equivalent, in terms of active pharmaceutical ingredient particle size, to FLONASE®. The testing was performed following a validated analytical method.

Mean values for the D50 and span (in number distribution) for each batch tested are shown in table 15, resulting from averaging the 25 replicates performed for each batch. The span is calculated from the PSD percentiles as per the following formula:

$$Span = \frac{D 90 - D 10}{D 50}$$

Both the span and the D50 are the variables suggested by the Draft Guidance on Fluticasone Propionate as the basis of the population bioequivalence (PBE) analysis included in the alternative approach to the comparative clinical endpoint bioequivalence (BE) study.

TABLE 15
Mean Values

| Time | XT5V | XT5W | B46J | SC3S | 6P2E | AY5S |
|---|---|---|---|---|---|---|
| D50 | 2.53 | 2.75 | 2.58 | 2.60 | 2.67 | 2.51 |
| (number) | | | | | | |
| Span | 0.83 | 0.85 | 0.88 | 0.77 | 0.80 | 0.89 |
| (number) | | | | | | |

The population bioequivalence statistical analysis was performed by following the Draft Guidance on Budesonide published by the FDA. The population bioequivalence criterion is defined as:

$$θ = \frac{{( {μ}_{T} - {μ}_{R} )}^{2} + ( {σ}_{T}^{2} - {σ}_{R}^{2} )}{max ( {σ}_{R}^{2} , {σ}_{T 0}^{2} )}$$

Where μ_{T} and μ_{S} are test and control product means on the log scale, σ²_{T} and σ²_{R} are test and control formulation variances on the log scape and is a regulatory constant with a value of 0.01. The population bioequivalence is defined as:

$$\frac{{( log ( 1.11 ) )}^{2} + 0.01}{{0.1}^{2}} .$$

The above describes a test mean at either 90% or 100% of the reference mean and a test variance that is twice the reference variance. The Draft Guidance on Budesonide published includes a method for calculation of PBE confidence intervals using the following linearized form of the criterion:

η=(μ_{T}−μ_{R})²+(σ_{T}²−σ_{R}²)−θ_{BE} max(σ_{R}²,σ_{T0}²)

In the above, population bioequivalence is determined when the upper 95% confidence interval is less than 0.

Tables 16 and 17 report the population bioequivalence results as suggested in the draft Guidance on Budesonide. The geometric mean and total variance of the test and reference samples are shown in Table 16.

TABLE 16
Population Bioequivalence Results

| | Geometric | Geometric | | | | |
|---|---|---|---|---|---|---|
| | mean | mean | | | | |
| Variable | (test) | (control) | GMR | σ_{T} | σ_{R} | σ_{T}/σ_{R} |
| D50 | 2.593 | 2.574 | 1.008 | 0.141 | 0.120 | 1.172 |
| (number) | | | | | | |
| Span | 0.846 | 0.810 | 1.044 | 0.131 | 0.147 | 0.889 |
| (number) | | | | | | |

The linearized point estimate and 95% upper confidence interval obtained by the PBE comparison of the test and control PSD results are shown in Table 17. All upper 95% confidence intervals are lower than 0 so population bioequivalence can be concluded for all metrics.

TABLE 17
Population Bioequivalence Results

| | | Linearized Point | 95% Upper |
|---|---|---|---|
| Variable | Scaling | Estimate | Confidence Interval |
| D50 (number) | Reference | −0.0248 | −0.0060 |
| Span (number) | Reference | −0.0480 | −0.0294 |

### CONCLUSION

In conclusion, the active pharmaceutical ingredient comparative particle size distribution testing and population bioequivalence data analysis performed in this study indicates that population bioequivalence can be concluded for all metrics evaluated (span and D50) comparing the test (preferred embodiment of the present invention) and control (FLONASE®) formulations.

What is claimed is:

1. A nasal spray formulation comprising:
at least one active pharmaceutical ingredient;
glycerin;
polyethylene glycol; and
dextrose, and
water
wherein the formulation retains at least about 3% water when exposed to about 80% relative humidity at a temperature of about 23° C. for about 750 minutes in a Dynamic Vapor Sorption (DVS) test.

2. The nasal spray formulation of claim 1, wherein the formulation has a pH between about 5 and about 7.

3. The nasal spray formulation of claim 1, wherein the formulation has an osmolarity between about 100 mOsmoles and about 800 mOsmoles.

4. The nasal spray formulation of claim 1, wherein the active pharmaceutical ingredient is present in an amount between about 0.005% w/w and about 0.2% w/w.

5. The nasal spray formulation of claim 1, wherein the active pharmaceutical ingredient is fluticasone propionate, fluticasone furoate, azelastine, oxymetazoline, xylometazoline, beclomethasone, mometasone, budesonide, salts and esters thereof, or a combination thereof.

6. The nasal spray formulation of claim 5, wherein the active pharmaceutical ingredient is fluticasone propionate.

7. The nasal spray formulation of claim 1, wherein the formulation is isotonic.

8. The nasal spray formulation of claim 7, wherein the formulation comprises an isotonicity adjusting agent being sodium chloride, dextrose, potassium chloride, or a combination thereof.

9. The nasal spray formulation of claim 8, wherein the isotonicity adjusting agent is sodium chloride.

10. The nasal spray formulation of claim 1, wherein glycerin is present in an amount between about 0.5% w/w and about 8% w/w.

11. The nasal spray formulation of claim 1, wherein polyethylene glycol is present in an amount between about 0.5% w/w and about 20% w/w.

12. The nasal spray formulation of claim 1, wherein the polyethylene glycol has an average molecular weight between about 200 and about 600.

13. The nasal spray formulation of claim 12, wherein the polyethylene glycol has an average molecular weight of about 400.

14. The nasal spray formulation of claim 1, wherein dextrose is present in an amount between about 0.3% w/w and about 7% w/w.

15. The nasal spray formulation of claim 1, wherein the formulation has a droplet size distribution of about 10% of droplets are less than about 18 μm, about 50% of droplets are between about 33 μm and about 51 μm, and about 90% of droplets are less than about 120 μm, at Tzero.

16. The nasal spray formulation of claim 1, wherein about 100% of the active pharmaceutical ingredient particles are less than about 10 μm at Tzero.

17. A method for treating the symptoms of allergic rhinitis whereby the nasal cavity feels moisturized, comprising administering the nasal spray formulation according to claim 1.

18. A method for treating the symptoms of allergic rhinitis whereby a gently soothing sensation is provided in the nasal cavity, comprising administering the nasal spray formulation according to claim 1.

19. A method of treating the symptoms of allergic rhinitis whereby the nasal cavity feels comfortable, comprising administering the nasal spray formulation according to claim 1.

20. The nasal spray formulation of claim 1, comprising:
the at least one active pharmaceutical ingredient in an amount between about 0.005% w/w and about 0.2% w/w,
glycerin in an amount between about 0.5% w/w and about 8% w/w,
polyethylene glycol having an average molecular weight of about 400 in an amount between about 0.5% w/w and about 20% w/w,
dextrose in an amount between about 0.3% w/w and about 7% w/w, and
water in an amount between about 80% w/w and about 99% w/w,
all weight percents being based on the total weight of the formulation.

21. A nasal spray formulation according to claim 20 wherein the active pharmaceutical ingredient comprises fluticasone propionate or fluticasone furoate.
